(19) United States
(12) Patent Application Publication
(10) Pub. No.: US 2018/0168465 A1
(43) Pub. Date: Jun. 21, 2018

(54) MEASUREMENT DEVICE AND MEASUREMENT METHOD

(71) Applicant: SEIKO EPSON CORPORATION, Tokyo (JP)

(72) Inventors: Akiko YAMADA, Shiojiri-shi (JP); Kohei YAMADA, Shiojiri-shi (JP)

(73) Assignee: SEIKO EPSON CORPORATION, Tokyo (JP)

(21) Appl. No.: 15/837,031

(22) Filed: Dec. 11, 2017

(30) Foreign Application Priority Data
Dec. 21, 2016 (JP) 2016-247702

(51) Int. Cl. A61B 5/026; A61B 5/02; A61B 5/0285

(52) U.S. Cl. CPC A61B 5/0261; A61B 5/0285; A61B 5/02007

(57) ABSTRACT

A measurement device includes a plurality of detection units that respectively include a light emitting unit which emits light to a measurement target site and a light receiving unit which generates detection signals corresponding to a light receiving level of the light emitted from the light emitting unit and passing through the inside of the measurement target site, and a selection unit that selects some of the detection signals in accordance with an intensity index indicating signal intensity of the respective detection signals, from the detection signals generated by the light receiving unit in each of the plurality of detection units.

### BACKGROUND

### 1. Technical Field

The present invention relates to a technique for measuring biological information.

### 2. Related Art

Various measurement techniques for noninvasively measuring biological information by irradiating a living body with light have been proposed in the related art. For example, JP-A-2004-201868 discloses a configuration in which blood flow velocity of an artery in a wrist is calculated based on a signal generated by an optical sensor disposed inside a wristband.

However, according to the technique disclosed in JP-A-2004-201868, in a case where a position of the wristband is misaligned with the artery, there is a possibility that a signal suitable for calculating the blood flow velocity (that is, a signal reflecting a light receiving level of light passing through the artery) may not be generated by the optical sensor.

### SUMMARY

An advantage of some aspects of the invention is to more accurately measure the biological information even in a case where a position of a measurement device is misaligned with a specific portion inside a measurement target site.

A measurement device according to a preferred aspect of the invention includes a plurality of detection units that respectively include a light emitting unit which emits light to a measurement target site and a light receiving unit which generates detection signals corresponding to a light receiving level of the light emitted from the light emitting unit and passing through the inside of the measurement target site, and a selection unit that selects some of the detection signals in accordance with an intensity index indicating signal intensity of the respective detection signals, from the detection signals generated by the light receiving unit in each of the plurality of detection units. According to this configuration, the detection signal is selected in accordance with the intensity index indicating the signal intensity, from the detection signals generated by the light receiving unit in each of the plurality of detection units. Therefore, for example, compared to a configuration having one detection unit included in a detection device, the biological information can be more accurately measured, even in a case where a position of the measurement device is misaligned with a specific portion (for example, an artery) inside the measurement target site.

In the preferred aspect of the invention, the measurement device may further include a calculation unit that calculates biological information relating to a blood flow inside the measurement target site, based on the detection signal selected by the selection unit. According to this configuration, the biological information relating to the blood flow of the measurement target site is calculated, based on the detection signal selected by the selection unit.

In the preferred aspect of the invention, the plurality of detection units may have the same distance between the light emitting unit and the light receiving unit. According to this configuration, the respective detection units have approximately the same depth at which the light reaching the light receiving unit from the light emitting unit passes through the inside of the measurement target site. Therefore, compared to a configuration in which the plurality of detection units have mutually different distances between the light emitting unit and the light receiving unit, the biological information can be more accurately measured, even in the case where the position of the measurement device is misaligned with the specific portion inside the measurement target site.

In the preferred aspect of the invention, the plurality of detection units may be installed along a first direction. According to this configuration, the plurality of detection units are installed along the first direction. Therefore, even in a case of a position relationship in which the specific portion (for example, a blood vessel) inside the measurement target site and the measurement device are misaligned with each other in the first direction, the light transmitted through the specific portion inside the measurement target site can be received by any one of the light receiving units.

In the preferred aspect of the invention, the light emitting unit and the light receiving unit may be located along the first direction in each of the plurality of detection units. According to this configuration, the light emitting unit and the light receiving unit are located along the first direction in each of the plurality of detection units. Therefore, for example, compared to a configuration in which the light emitting unit and the light receiving unit are located along a direction intersecting the first direction in each of the plurality of detection units, if the measurement device has the same number of installed detection units, the biological information can be much more accurately measured, even in the case of the position relationship in which the specific portion inside the measurement target site and the measurement device are misaligned with each other in the first direction.

In the preferred aspect of the invention, the light emitting unit and the light receiving unit may be located along a second direction intersecting the first direction in each of the plurality of detection units. According to this configuration, the light emitting unit and the light receiving unit are located along the second direction intersecting the first direction in each of the plurality of detection units. Therefore, compared to a configuration in which the light emitting unit and the light receiving unit are located along the first direction in each of the plurality of detection units, the more advantageous effect is achieved in that the detection unit can be more densely installed in the first direction.

In the preferred aspect of the invention, the first direction may be a direction intersecting an artery inside the measurement target site. According to this configuration, the plurality of detection units are arranged in the direction intersecting the artery inside the measurement target site. Therefore, there is an increasing possibility that any one of the plurality of detection units may be located on the artery.

In the preferred aspect of the invention, the measurement device may further include a belt for supporting the plurality of detection units with respect to the measurement target site, and the first direction may be a circumferential direction of the belt. According to this configuration, the plurality of detection units are arranged in the circumferential direction of the belt. Therefore, the detection signals are generated from the plurality of detection units arranged on a straight line in a direction intersecting a width direction of the belt.

In the preferred aspect of the invention, the light emitted to the measurement target site from the respective light emitting units may be coherent light, and a distance between the light emitting unit and the light receiving unit in each of the plurality of detection units may be longer than 0.5 mm, and may be shorter than 3 mm. According to this configuration, the distance between the light emitting unit and the light receiving unit in each of the plurality of detection units is longer than 0.5 mm and shorter than 3 mm. Therefore, compared to a configuration in which the distance between the light emitting unit and the light receiving unit in each of the plurality of detection units is shorter than 0.5 mm and is longer than 3 mm, the detection signal having a higher S/N ratio can be generated.

In the preferred aspect of the invention, each of the plurality of detection units may include the plurality of light receiving units having the same distance from the light emitting unit and the light emitting unit. According to this configuration, the detection signal is generated by each of the plurality of light receiving units having the same distance from the light emitting unit and the light emitting unit. Therefore, compared to a configuration in which the light emitting units are arranged for the plurality of light receiving units in a one-to-one relationship, power saving and downsizing of the device can be achieved.

A measurement method according to a preferred aspect of the invention is a measurement method of measuring biological information relating to a blood flow inside a measurement target site by using a plurality of detection units respectively including a light emitting unit that emits light to the measurement target site and a light receiving unit that generates detection signals corresponding to a light receiving level of the light emitted from the light emitting unit and passing through the inside of the measurement target site. The measurement method includes causing a computer to select some of the detection signals in accordance with an intensity index indicating signal intensity of the respective detection signals, from the detection signals generated by the light receiving unit in each of the plurality of detection units, and causing the computer to calculate the biological information, based on the selected detection signal. According to this configuration, the same operation and advantageous effect as those according to the measurement device of the invention can be realized.

### BRIEF DESCRIPTION OF THE DRAWINGS

The invention will be described with reference to the accompanying drawings, wherein like numbers reference like elements.

FIG. 1 is a side view of a measurement device according to a first embodiment of the invention.

FIG. 2 is a configuration diagram focusing on a function of the measurement device.

FIG. 3 is a view for describing a position of each detection unit with respect to an artery.

FIG. 4 is a graph illustrating a relationship between a distance between a light emitting unit emitting coherent light with irradiation intensity of 3 mW/cm² and a light receiving unit and an S/N ratio of a detection signal.

FIG. 5 is a graph illustrating a relationship between a distance between a light emitting unit emitting coherent light with irradiation intensity of 1 mW/cm² and a light receiving unit and an S/N ratio of a detection signal.

FIG. 6 is a flowchart of an operation of a control device.

FIG. 7 is a view for describing a position of each detection unit with respect to an artery according to a second embodiment.

FIG. 8 is a graph illustrating a relationship between a distance from a central axis of the artery to the detection unit and an intensity index of a detection signal.

FIG. 9 is a view for describing each detection unit according to a modification example.

FIG. 10 is a view for describing each detection unit according to a modification example.

FIG. 11 is a view for describing a position of each detection unit according to a modification example.

### DESCRIPTION OF EXEMPLARY EMBODIMENTS

### First Embodiment

FIG. 1 is a side view of a measurement device 100 according to a first embodiment of the invention. The measurement device 100 is a measuring instrument for calculating biological information relating to a blood flow of a subject, and is mounted on a site to be measured (hereinafter, referred to as a “measurement target site”) M of a body of the subject. In the first embodiment, a wrist of the subject will be described as an example of the measurement target site M. Specifically, the measurement device 100 calculates the biological information relating to the blood flow of an artery A (radial artery and ulnar artery) present inside the measurement target site M. In the first embodiment, a blood flow rate of the artery A will be described as an example of the biological information relating to the blood flow.

The measurement device 100 according to the first embodiment is a wristwatch-type portable instrument including a belt 14 wrapped around the measurement target site M and a housing 12 fixed to the belt 14. The belt 14 is wrapped around the wrist serving as an example of measurement target site M, thereby enabling the measurement device 100 to be mounted on the wrist of the subject. The measurement device 100 comes into contact with a surface of the wrist of the subject.

Hereinafter, a direction intersecting (typically orthogonal to) the artery A in FIG. 1 is referred to as a first direction x, and a direction intersecting (typically orthogonal to)) the first direction x is referred to as a second direction y. As illustrated in FIG. 1, the first direction x is a circumferential direction L of the belt 14, and can be regarded as a direction along a longitudinal direction of the belt 14. The second direction y is a direction parallel to an extending direction of the artery A, and can be regarded as a direction along a width direction W of the belt 14. The width direction of the belt 14 is a transverse direction of the belt 14 having a strip shape, and can be regarded as a direction of a central axis J of a cylinder having the belt 14 as a side surface. One side in the first direction x is referred to as an x1-side, and a side opposite to the x1-side is referred to as an x2-side. One side in the second direction y is referred to as a y1-side, and a side opposite to the y1-side is referred to as a y2-side.

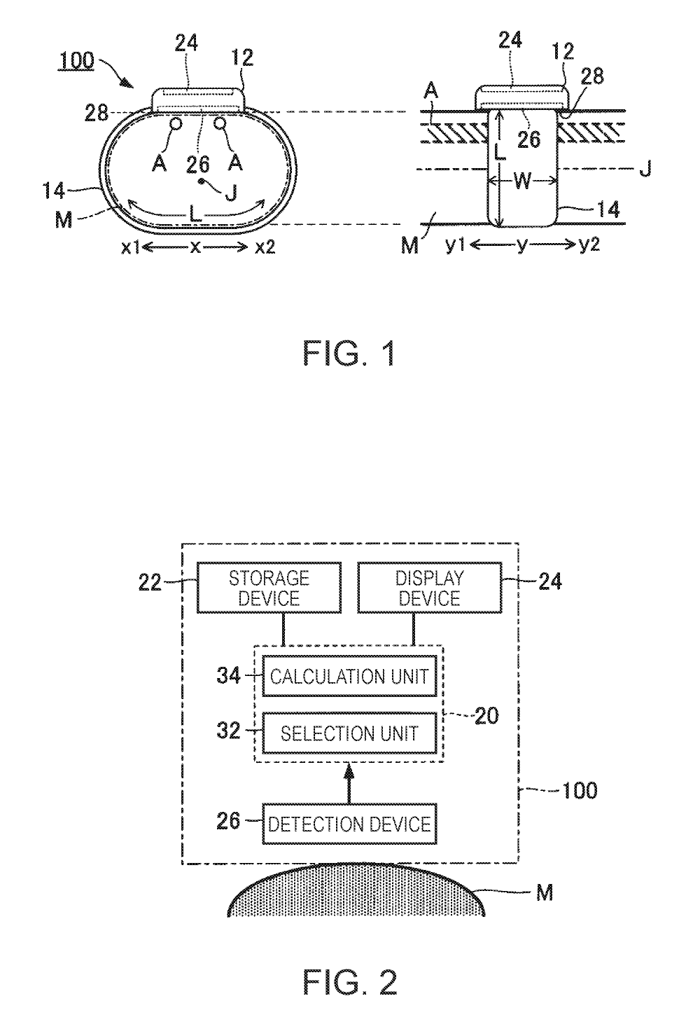

FIG. 2 is a configuration diagram focusing on a function of the measurement device 100. As illustrated in FIG. 2, the measurement device 100 according to the first embodiment includes a control device 20, a storage device 22, a display device 24, and a detection device 26. The control device 20 and the storage device 22 are installed inside a housing 12. As illustrated in FIG. 1, the display device 24 (for example, a liquid crystal display panel) is installed on a surface (for example, a surface on a side opposite to the measurement target site M) of the housing 12, and displays various images including measurement results under the control of the control device 20.

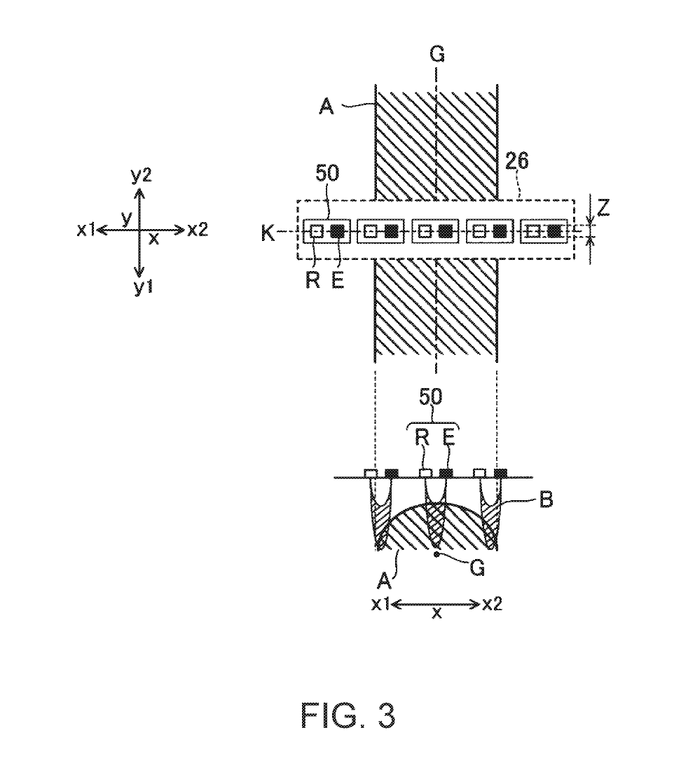

The detection device 26 in FIG. 2 is a sensor module which generates a plurality of detection signals corresponding to a state of the measurement target site M. For example, the detection device 26 is installed on a surface (hereinafter, referred to as a “detection surface”) 28 facing the measurement target site M in the housing 12. The detection device 26 is supported with respect to the measurement target site M by the belt 14. The detection surface 28 is a plane or a curved surface. The detection device 26 according to the first embodiment includes a plurality of detection units 50, as illustrated in FIG. 3. Each of the plurality of detection units 50 includes a light emitting unit E and a light receiving unit R, and generates a detection signal corresponding to a state of the measurement target site M.

The light emitting unit E emits light to the measurement target site M. The light emitting unit E according to the first embodiment is a light emitting element which emits coherent light (that is, laser light) having high coherence. As the light emitting element which emits the laser light, a surface emitting laser (VCSEL; vertical cavity surface emitting laser), a photonic crystal laser, or a semiconductor laser can be employed. The respective light emitting units E simultaneously emit the light to the measurement target site M. However, a light emitting diode (LED) can be used as the light emitting unit E. The plurality of light emitting units E have the same irradiation intensity (for example, 3 mW/cm² or smaller) of the light emitted by the respective light emitting units E according to the first embodiment.

The light emitted from the light emitting unit E is incident on the measurement target site M, and repeatedly reflected and scattered inside the measurement target site M. Thereafter, the light exits to the detection surface 28 side, and reaches the light receiving unit R. That is, the light emitting unit E and the light receiving unit R function as a reflection type optical sensor.

The light receiving unit R generates a detection signal corresponding to a light receiving level of the light passing through the inside of the measurement target site M. For example, a photoelectric conversion element such as a photo diode (PD) which receives the light by using a light receiving surface facing the measurement target site M is suitably used as the light receiving unit R. For example, a shape of the light receiving surface of the light receiving unit R is a square of 0.2 mm. For example, each of the detection units 50 includes a drive circuit for driving the light emitting unit E by supplying a drive current and an output circuit (for example, an amplifier circuit and an A/D converter) for performing amplifying and A/D converting on an output signal of the light receiving unit R. However, each circuit is omitted in the illustration of FIG. 3.

The artery A inside the measurement target site M repeatedly expands and contracts with a cycle equivalent to a pulsation. The blood flow rate inside the blood vessel fluctuates when the artery A expands and contracts. Accordingly, the detection signal generated by the respective light receiving units R in response to the light receiving level transmitted from the measurement target site M is a pulse wave signal including a periodic fluctuation component corresponding to fluctuations of the blood flow rate of the blood vessel of the measurement target site M.

As illustrated in FIG. 3, the plurality of detection units 50 according to the first embodiment are installed along the first direction x, that is, so as to intersect the artery A (having a diameter of approximately 2 to 3 mm). Specifically, each of the plurality of detection units 50 is installed at a different location on a straight line K parallel to the first direction x. The plurality of detection units 50 are installed at equal intervals along the first direction x. However, density of the plurality of detection units 50 can be changed. For example, in an arrangement of the plurality of detection units 50, the plurality of detection units 50 closer to a central side portion of the arrangement can be more densely arranged compared to both end sides of the arrangement. The description that the detection unit 50 is located on the straight line K means that the straight line K is located inside a range Z (range from an end portion on the y1-side to an end portion on the y2-side in the second direction y) where the light receiving unit R and the light emitting unit E of the detection unit 50 are present.

The light emitting unit E and the light receiving unit R in each of the plurality of detection units 50 are located along the first direction x. Specifically, the center of the light emitting unit E and the center of the light receiving unit R are located on the straight line K. In the respective detection units 50, the light emitting unit E is located on the x2-side on the straight line K, and the light receiving unit R is located on the x1-side on the straight line K. All of the detection units 50 have the same distance between the light emitting unit E and the light receiving unit R in the respective detection units 50. The distance between the light emitting unit E and the light receiving unit R means a distance between the respective centers of the light emitting unit E and the light receiving unit R. In the detection device 26 according to the first embodiment, as illustrated in FIG. 3, the light emitting unit E and the light receiving unit R are alternately arranged in the first direction x across the plurality of detection units 50.

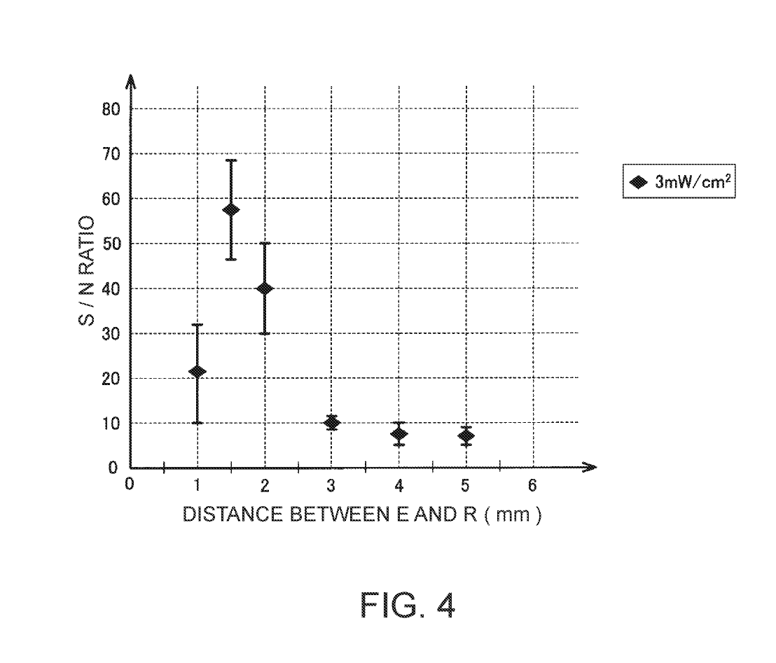

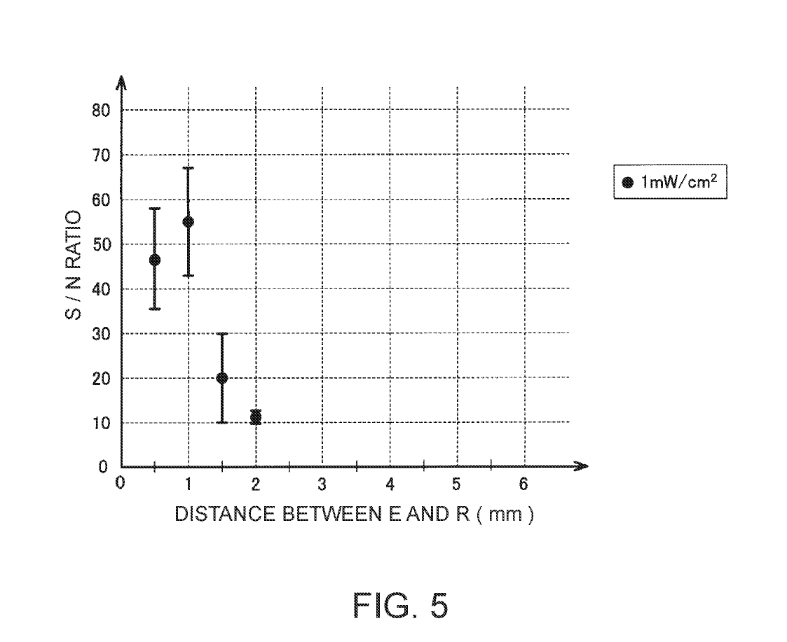

FIGS. 4 and 5 are graphs illustrating a relationship between the distance between the light emitting unit E and the light receiving unit R in the respective detection units 50 and an S/N ratio of the detection signal generated by the light receiving unit R. FIG. 4 illustrates a case where the coherent light is emitted using irradiation intensity of 3 mW/cm². FIG. 5 illustrates a case where the coherent light is emitted using the irradiation intensity of 1 mW/cm². The S/N ratio represents an intensity ratio between a signal component and a noise component, and means that the detection signal more suitable for calculating the biological information is generated as the S/N ratio is higher. As illustrated in FIGS. 4 and 5, the S/N ratio shows a high value in a case where the distance between the light emitting unit E and the light receiving unit R is in a range of 0.5 mm to 3 mm. The S/N ratio is more conspicuous in a case where the distance is in a range of 1 mm to 1.5 mm. Therefore, in the first embodiment, the distance between the light emitting unit E and the light receiving unit R is set to be in the range of 0.5 mm to 3 mm and preferably set to be in the range of 1 mm to 1.5 mm. As a result of adopting the above-described configuration, it is possible to generate the detection signal having the high S/N ratio. The above-described configuration is particularly effective in a case where the light emitted from the light emitting unit E is the coherent light.

The control device 20 illustrated in FIG. 2 is an arithmetic processing device such as a central processing unit (CPU) and a field-programmable gate array (FPGA), and controls the overall measurement device 100. For example, the storage device 22 is configured to include a nonvolatile semiconductor memory, and stores a program executed by the control device 20 and various data items used by the control device 20. The control device 20 according to the first embodiment executes the program stored in the storage device 22 so as to fulfill a plurality of functions (the selection unit 32 and the calculation unit 34) for calculating the blood flow rate of the artery A. A configuration can be adopted in which the function of the control device 20 is distributed to a plurality of integrated circuits, or a configuration can be adopted in which the functions of the control device 20 are partially or entirely realized by a dedicated electronic circuit. Although the control device 20 and the storage device 22 are illustrated as separate elements in FIG. 2, the control device 20 including the storage device 22 can be realized by an application specific integrated circuit (ASIC), for example.

The selection unit 32 selects the detection signal to be used for calculating the blood flow rate, based on the detection signal generated by the light receiving unit R in each of the plurality of detection units 50. The selection unit 32 according to the first embodiment selects some of the detection signals in accordance with an index indicating signal intensity (hereinafter, referred to as an “intensity index”) of each detection signal, from the detection signals generated by the light receiving unit R in each of the plurality of detection units 50. In the first embodiment, the S/N ratio of the detection signal will be described as an example of the intensity index.

Here, the intensity indexes of the detection signals generated by the respective detection units 50 are different from each other at positions of the detection units 50 which generate the detection signals with respect to the artery A. FIG. 3 illustrates a range (hereinafter, referred to as a “propagation range”) B in which the light reaching the light receiving unit R from the light emitting unit E propagates inside the measurement target site M. The propagation range B means a range (so-called banana shape) in which the light having intensity exceeding a predetermined value is distributed. As illustrated in FIG. 3, the propagation range B of the detection unit 50 located on a central axis G (straight line parallel to the second direction y) of the artery A is likely to be overlapped with an extending range of the artery A in a plan view, compared to the propagation range B of the detection unit 50 located at the position separated from the central axis G. That is, the intensity index of the detection signal generated from the detection unit 50 located closer to the central axis G in a plan view becomes higher, and the intensity index of the detection signal generated from the detection unit 50 located farther from the central axis G in a plan view becomes lower. In other words, the detection signal having the higher intensity index is generated by more receiving the light transmitted through the artery A. As described above, the selection unit 32 according to the first embodiment selects one detection signal whose intensity index is highest (that is, the light emitted from the light emitting unit E passes through the utmost inside of the artery A), from the detection signals generated by the respective detection units 50. In other words, the selection unit 32 selects the detection signal generated by the light receiving unit R located closest to the artery A from the plurality of light receiving units R.

Specifically, the selection unit 32 calculates the intensity index for the respective detection signals, and selects the detection signal having the highest intensity index from the plurality of detection signals. A method of calculating the intensity index is optionally used. For example, the selection unit 32 calculates the intensity index, based on an average of amplitudes of a plurality of cycles (for example, ten cycles) of the detection signal.

The calculation unit 34 calculates a blood flow rate Q of the artery A, based on the detection signal selected by the selection unit 32. A known technique can optionally be employed for calculating the blood flow rate Q. For example, the calculation unit 34 uses Equation (1) below so as to calculate the blood flow rate Q. The reference numeral fd represents a frequency of a beat signal generated by interference between the light scattered from a stationary tissue and the light scattered from a moving blood cell. The reference numeral I represents light receiving intensity of the light receiving unit R. The reference numeral Φ(fd) represents a power spectrum of the detection signal, and is calculated using Fast Fourier Transform (FFT), for example. The calculation unit 34 causes the display device 24 to display the calculated blood flow rate Q.

$$Q = \frac{\int {f}_{d} \cdot Φ ( {f}_{d} ) {df}_{d}}{{I}^{2}} \qquad ( 1 )$$

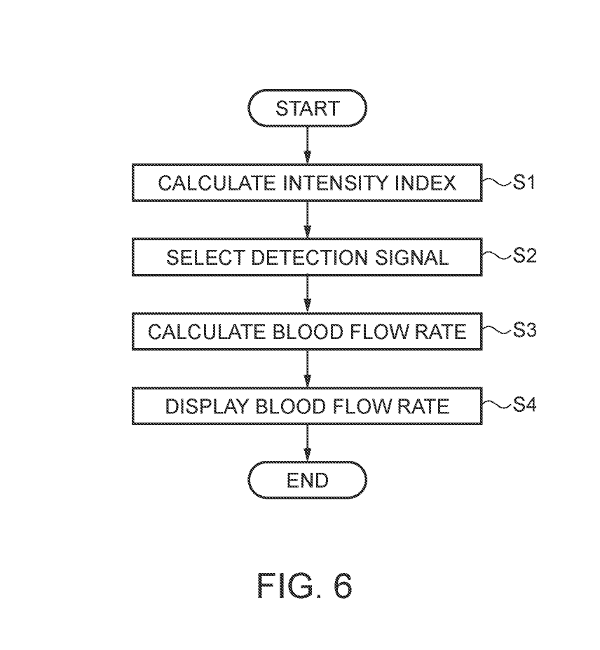

FIG. 6 is a flowchart of a process operation of the control device 20. The process in FIG. 6 starts with a measurement start instruction (program activation) made from a subject as a trigger. The selection unit 32 calculates the intensity index for the detection signal generated by the light receiving unit R in each of the plurality of detection units 50 (S1). The selection unit 32 selects the detection signal having the calculated highest intensity index from the plurality of detection signals (S2). The calculation unit 34 calculates the blood flow rate Q, based on the detection signal specified by the selection unit 32 (S3). The calculation unit 34 causes the display device 24 to display the calculated blood flow rate Q (S4). The processes from Step S1 to Step S4 are repeatedly performed at predetermined intervals.

Here, for example, in a case of adopting a configuration having one detection unit 50 included in the detection device 26, there is an individual difference in the position of the artery A inside the living body, and the user is less likely to find the position of the artery A inside the measurement target site M. Accordingly, there is a possibility that the position of the detection unit 50 may be apart from the central axis G of the artery A. Consequently, a problem arises in that a suitable detection signal reflecting the light receiving level of light passing through the artery A cannot be generated. In contrast, in the first embodiment, the detection signal in accordance with the intensity index is selected from the plurality of the detection signals generated by the respective detection units 50. Accordingly, even in a case where the position of the measurement device 100 is misaligned with the artery A, it is possible to select the suitable detection signal reflecting the light receiving level of the light passing through the artery A. Therefore, the first embodiment has an advantageous effect in that the blood flow rate Q of the artery A can be more accurately calculated using the suitable detection signal reflecting the light receiving level of the light passing through the artery A.

### Second Embodiment

A second embodiment according to the invention will be described. In each configuration described below as an example, the reference numerals used in describing the first embodiment will be used for elements whose operation or function is the same as that according to the first embodiment, and each detailed description thereof will be appropriately omitted.

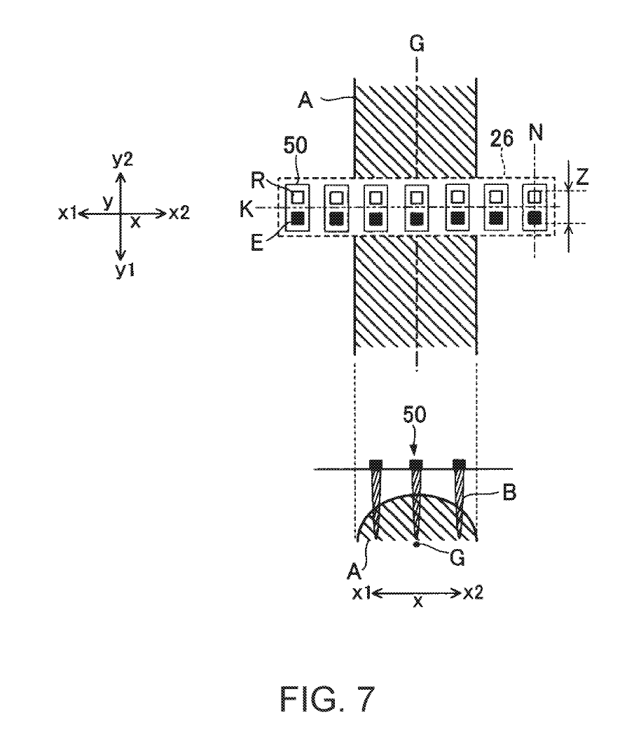

In the first embodiment, the light emitting unit E and the light receiving unit R in each of the plurality of detection units 50 are located along the first direction x. In contrast, in the second embodiment, as illustrated in FIG. 7, the light emitting unit E and the light receiving unit R in each of the plurality of detection units 50 are located along the second direction y intersecting the first direction x.

Similarly to the first embodiment, the detection device 26 according to the second embodiment includes a plurality of detection units 50. Similarly to the first embodiment, the plurality of detection units 50 according to the second embodiment include the light emitting unit E and the light receiving unit R, and are respectively installed at different positions on the straight line K parallel to the first direction x. As illustrated in FIG. 7, the light emitting unit E and the light receiving unit R in each of the plurality of detection units 50 are located along the second direction y. Specifically, the center of the light emitting unit E and the center of the light receiving unit R are located on a straight line N parallel to the second direction y (the central axis G). In each detection unit 50, the light emitting unit E is located on the y1-side on the straight line N, and the light receiving unit R is located on the y2-side on the straight line N. All of the detection units 50 have the same distance between the light emitting unit E and the light receiving unit R in each detection unit 50.

In the second embodiment, it is also understood that the propagation range B of the detection unit 50 located on the central axis G of the artery A is likely to be overlapped with the extending range of the artery A in a plan view, as illustrated in FIG. 7, compared to the propagation range B of the detection unit 50 located at a distance separated from the central axis G. Therefore, similarly to the first embodiment, the selection unit 32 according to the second embodiment also selects the detection signal having the highest intensity index, from the detection signals generated by the light receiving unit R in each of the plurality of detection units 50. Similarly to the first embodiment, the calculation unit 34 according to the second embodiment calculates the blood flow rate Q of the artery A, based on the detection signals selected by the selection unit 32. In the second embodiment, the same advantageous effect as that according to the first embodiment can be realized.

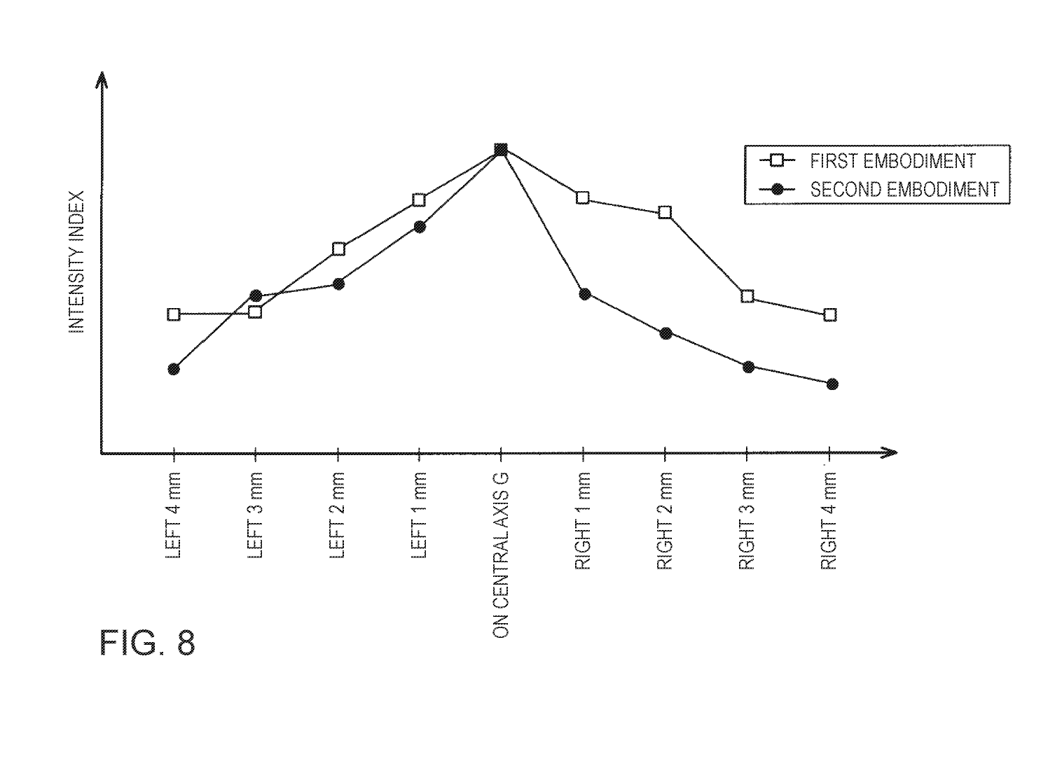

FIG. 8 is a graph illustrating a relationship between the distance from the central axis G of the artery A to the detection unit 50 (midpoint of a line segment connecting the light emitting unit E and the light receiving unit R) and the intensity index of the detection signal. The intensity indexes of the detection signals generated by the respective detection units 50 installed by being misaligned as far as the distance on the horizontal axis, based on the detection unit 50 located on the central axis G are illustrated about a configuration according to the first embodiment and a configuration according to the second embodiment. In the configuration according to the first embodiment and the configuration according to the second embodiment, a case is assumed where the detection units 50 are installed along the first direction x at each interval of 1 mm to the left and right, based on the detection unit 50 located on the central axis G. As described above, the first embodiment adopts the configuration in which the light emitting unit E and the light receiving unit R of the respective detection units 50 are located along the first direction x, and the second embodiment adopts the configuration in which the light emitting unit E and the light receiving unit R of the respective detection units 50 are located along the second direction y.

As illustrated in FIG. 8, in both configurations of the first embodiment and the second embodiment, the intensity index of the detection signal generated by the detection unit 50 located on the central axis G is the highest. It is understood that the intensity index of the detection signal generated by the detection unit 50 is lowered as the position of the detection unit 50 is separated from the central axis G to the left and right. However, in the configuration of the first embodiment, compared to the configuration of the second embodiment, the intensity index is higher even in a case where the position of the detection unit 50 is misaligned with the central axis G to the left or right. As can be understood from the above description, in a case where the respective detection units 50 are installed at the same position in the first embodiment and in the second embodiment, compared to the configuration of the second embodiment, the configuration of the first embodiment can much more accurately calculate the biological information even in a case of the position relationship in which the artery A and the measurement device 100 are misaligned with each other in the first direction x. However, in the configuration of the second embodiment in which the light emitting unit E and the light receiving unit R of the respective detection units 50 are located along the second direction y, compared to the configuration of the first embodiment, the more advantageous effect is achieved in that the detection units 50 can be more densely installed along the first direction x.

### Modification Example

Each embodiment described above can be modified in various ways. Hereinafter, specific modification aspects will be described. Two or more optionally selected aspects from the following examples can be appropriately combined with each other.

(1) In each of the above-described embodiments, the S/N ratio has described as an example of the intensity index. However, the intensity index is not limited to the above-described example. For example, a configuration can be adopted in which the signal intensity itself of the detection signal is set as an example of the intensity index. A representative value (average value or maximum value) of the intensity within a specific range (for example, one cycle or a plurality of cycles) can be used as the intensity index.

(2) In each of the above-described embodiments, the blood flow rate Q is calculated as the biological information relating to the blood flow inside the measurement target site M. However, a type of the biological information relating to the blood flow is not limited to the above-described example. For example, a configuration can be adopted in which pulse wave velocity (PWV) or blood pressure is calculated as the biological information relating to the blood flow inside the measurement target site M.

(3) In each of the above-described embodiments, the selection unit 32 selects the detection signal having the highest intensity index from the detection signals generated by the light receiving unit R in each of the plurality of detection units 50. However, the number of the detection signals selected by the selection unit 32 is not limited to one. The selection unit 32 can select a plurality of detection signals from the respective detection signals. For example, the selection unit 32 selects a predetermined number of detection signals located high in a descending order of the intensity indexes. A configuration can also be preferably adopted in which the selection unit 32 selects the detection signal having the highest intensity index and the detection signal generated by each of the two detection units 50 installed at the position close from the detection unit 50 generating the detection signal having the highest intensity index. For example, the calculation unit 34 calculates a weighted average by using the average of the biological information calculated for each of the plurality of detection signals selected by the selection unit 32, or by using a weighting value according to the intensity index. As is understood from the above description, the selection unit 32 is comprehensively expressed as an element that selects some of the detection signals in accordance with the intensity index indicating the signal intensity of each detection signal, from the detection signals generated by the light receiving unit R in each of the plurality of detection units 50.

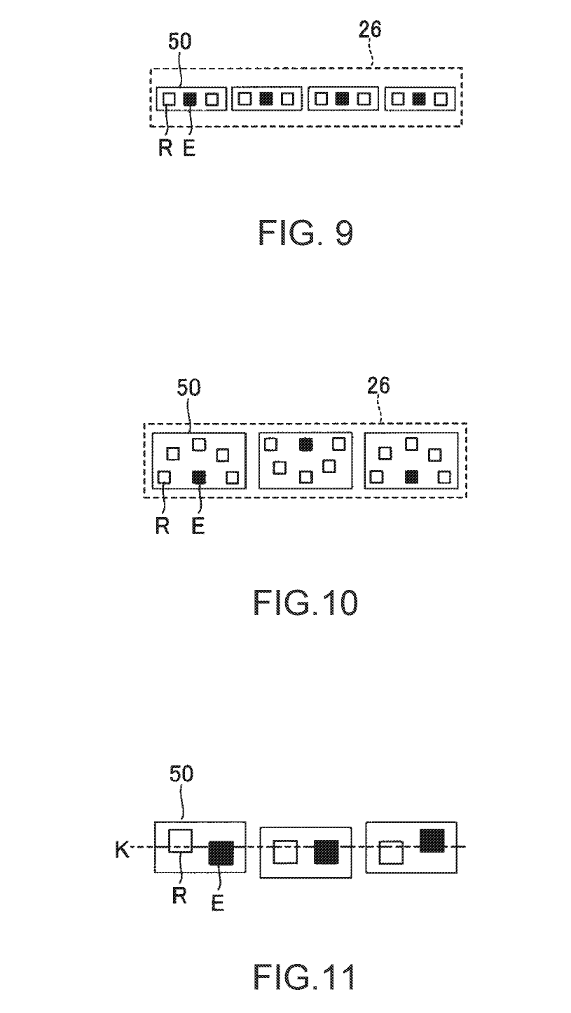

(4) In each of the above-described embodiments, a configuration has been described in which each detection unit 50 includes one light emitting unit E and one light receiving unit R. However, a configuration can be adopted in which each detection units 50 includes a plurality of light receiving units R. The plurality of light receiving units R included in the detection unit 50 have the same distance from the light emitting unit E. For example, as illustrated in FIG. 9, a configuration can be adopted in which each detection unit 50 includes one light emitting unit E and two light receiving units R interposing the light emitting unit E therebetween. Alternatively, as illustrated in FIG. 10, a configuration can be adopted in which each detection unit 50 includes one light emitting unit E and the plurality of light receiving units R located on the circumference centered on the light emitting unit E. In FIG. 9, the configuration has been described in which one light emitting unit E and two light receiving units R are arranged in the first direction x. However, one light emitting unit E and two light receiving units R can be arranged in the second direction y. The selection unit 32 selects some of the detection signals according to the intensity index from the detection signals generated by the plurality of light receiving units R included in each detection unit 50. According to the configuration in which the detection unit 50 includes the plurality of light receiving units R located as far as the same distance from the light emitting unit E, compared to a configuration in which the light emitting units E are disposed for the plurality of light receiving units R in a one-to-one relationship, power saving and downsizing of the device can be achieved. The distance between the light emitting units E increases. Accordingly, the influence received by the light receiving unit R from the light emitted from the light emitting unit E of the other detection unit 50 can be reduced.

(5) In each of the above-described embodiments, the measurement device 100 includes the calculation unit 34 that calculates the biological information relating to the blood flow inside the measurement target site M. However, the calculation unit 34 can be omitted from the measurement device 100. In the above-described configuration, the measurement device 100 transmits the selected detection signal to an external device (for example, a smartphone) capable of communicating with the measurement device 100. The external device calculates the biological information from the received detection signal. According to the above-described configuration, even in a case where the position of the measurement device 100 is misaligned with the specific portion inside the measurement target site M, an advantageous effect can also be achieved in that the biological information can be more accurately measured.

(6) In each of the above-described embodiments, the plurality of detection units 50 are installed along the first direction x. However, the position for installing the plurality of detection units 50 is not limited to the above-described example. For example, the plurality of detection units 50 can be arranged in a plane shape (for example, in a matrix shape extending in the first direction x and the second direction y). However, according to the configuration in which the plurality of detection units 50 are installed along the first direction x, even in a case of a position relationship in which the specific portion inside the measurement target site M and the measurement device 100 are misaligned with each other in the first direction x, the light transmitted through the specific portion can be received by any one of the light receiving units R.

(7) In each of the above-described embodiments, the direction intersecting the artery A inside the measurement target site M has been described as an example of the first direction x. However, for example, a direction parallel to the artery A can be set as the first direction x. However, according to the configuration where the direction intersecting the artery A inside the measurement target site M is set as the first direction x, there is an increasing possibility that any one of the plurality of detection units 50 may be located on the artery A. Therefore, the biological information relating to the blood flow of the artery A can be more accurately calculated.

(8) In each of the above-described embodiments, a configuration has been described in which the center of the light emitting unit E and the light receiving unit R in each of the plurality of detection units 50 is located on the straight line K (straight line N in the second embodiment). However, the position on the straight line K of the light emitting unit E and the light receiving unit R is not limited to the above-described example. For example, as illustrated in FIG. 11, even in a configuration in which the center of the light emitting unit E and the light receiving unit R is not located on the straight line K, if both of these only partially overlap the straight line K in a plan view, it can be regarded that the light emitting unit E and the light receiving unit R are located on the straight line K.

(9) In the first embodiment, in each detection unit 50, the light emitting unit E is located on the x2-side on the straight line K, and the light receiving unit R is located on the x1-side on the straight line K. However, a position relationship between the light emitting unit E and the light receiving unit R in each detection unit 50 is not limited to the above-described example. For example, a configuration can be adopted in which the light emitting unit E is located on the x1-side on the straight line K in each detection unit 50, and the light receiving unit R is located on the x2-side on the straight line K. Alternatively, a configuration can be adopted in which each detection unit 50 has a mutually different position relationship between the light emitting unit E and the light receiving unit R.

(10) In the second embodiment, in each detection unit 50, the light emitting unit E is located on the y1-side on the straight line N, and the light receiving unit R is located on the y2-side on the straight line N. However, the position relationship between the light emitting unit E and the light receiving unit R is not limited to the above-described examples. For example, a configuration can be adopted in which the light emitting unit E is located on the y2-side on the straight line N in each detection unit 50, and the light receiving unit R is located on the y1-side on the straight line N. Alternatively, a configuration can be adopted in which each detection unit 50 has a mutually different position relationship between the light emitting unit E and the light receiving unit R.

(11) In each of the above-described embodiments, a configuration has been described in which all of the detection units 50 have the same distance between the light emitting unit E and the light receiving unit R in each detection unit 50. However, a configuration can be adopted in which each detection unit 50 has a mutually different distance between the light emitting unit E and the light receiving unit R. However, according to the configuration in which all of the detection units 50 have the same distance between the light emitting unit E and the light receiving unit R in each detection unit 50, the respective detection units 50 have approximately the same depth (that is, the depth of the propagation range B) at which the light reaching the light receiving unit R from the light emitting unit E passes through the inside of the measurement target site M. Therefore, the intensity index of the detection signal generated by the light receiving unit R closest to the artery A in the plurality of light receiving units R is highest. As is understood from the above description, according to the configuration in which the respective detection units 50 have the same distance between the light emitting unit E and the light receiving unit R, compared to a configuration in which each detection unit 50 has the mutually different distance between the light emitting unit E and the light receiving unit R, even in a case where the position of the measurement device 100 is misaligned with the artery A inside the measurement target site M, the biological information can be much more accurately measured.

(12) In each of the above-described embodiments, the signal used in selecting the detection signal is also used for calculating the blood flow rate Q. However, the detection unit 50 can separately generate the detection signal to be used for calculating the blood flow rate Q. For example, after the detection signal is selected by the selection unit 32, light emission of the detection unit 50 other than the detection unit 50 which generates the selected detection signal is stopped. The detection unit 50 which generates the selected detection signal generates the detection signal to be used for calculating the blood flow rate Q. The calculation unit 34 calculates the blood flow rate Q by using the detection signal generated by the detection unit 50. According to the above-described configuration, the blood flow rate Q can be calculated using the detection signal which is less affected by the light emitted from the light emitting unit R of the other detection unit 50. However, according to the configuration in which the signal used for selecting the detection signal is also used for calculating the blood flow rate Q, power saving can be achieved.

(13) In each of the above-described embodiments, a configuration has been described in which the respective light emitting units E simultaneously emit the light to the measurement target site M. However, a configuration can be adopted in which the respective light emitting units E emit the light in a time division manner. According to the configuration in which the respective light emitting units E emit the light in the time division manner, an advantageous effect is achieved in that the light receiving unit R is less likely to receive the influence of the light emitted from the light emitting unit E of the other detection unit 50.

(14) In each of the above-described embodiments, a single measurement device 100 generates the plurality of detection signals, selects some of the detection signals from the plurality of detection signals, and calculates the biological information. However, the function of the measurement device 100 in the above-described respective embodiments can be realized by a plurality of devices. For example, the detection signal can be selected and the biological information can be calculated in such a way that a terminal device capable of communicating with the detection device 26 which generates the plurality of detection signals is used as the measurement device 100. Specifically, the plurality of detection signals generated by the detection device 26 are transmitted to the terminal device. The terminal device selects some of the detection signal from the plurality of detection signals received from the detection device 26, and calculates the biological information. As is understood from the above-described example, the detection device 26 and the control device 20 may be configured to be separate from each other.

A configuration may be adopted in which any one or both the selection unit 32 and the calculation unit 34 are disposed in the terminal device (for example, a configuration realized by an application executed by the terminal device). As is understood from the above description, the measurement device 100 can also be realized by a plurality of devices configured to be separate from each other.

(15) In each of the above-described embodiments, the measurement device 100 configured to include the belt 14 and the housing 12 has been described. However, a specific form of the measurement device 100 is optionally employed. For example, it is possible to employ the measurement device 100 of any desired type such as a patch type which can be attached to a body of a subject, an earring type which can be mounted on an auricle of the subject, a finger wearable type (for example, a claw type or a ring type) which can be mounted on a fingertip of the subject, and a head mount type which can be mounted on a head of the subject. A configuration can be adopted in which the belt 14 and the measurement device 100 are integrated with each other. However, for example, in a state where the measurement device 100 of the finger wearable type is mounted on the fingertip, it is assumed that the measurement device 100 may interfere with everyday activities. Therefore, from a viewpoint of constantly generating the detection signal without interfering with everyday activities, the measurement device 100 having the above-described form which can be mounted on the wrist of the subject by using the belt 14 is particularly preferable. The measurement device 100 having a form in which the measurement device 100 is mounted on (for example, externally attached to) various electronic devices such as a wristwatch can also be realized.

(16) The invention can also be specified as an operation method (measurement method) of the measurement device 100. Specifically, the measurement method according to a preferred aspect of the invention is as follows. The biological information relating to the blood flow inside the measurement target site M is measured using the plurality of detection units 50 respectively including the light emitting unit E that emits the light to the measurement target site M and the light receiving unit R that generates the detection signal according to the light receiving level of the light emitted from the light emitting unit E and passing through the inside of the measurement target site M. The measurement method includes causing a computer to select some of the detection signals in accordance with the intensity index indicating the signal intensity of the respective detection signals, from the detection signals generated by the light receiving unit R in each of the plurality of detection units 50, and causing the computer to calculate the biological information, based on the selected detection signal.

The entire disclosure of Japanese Patent Application No. 2016-247702 is hereby incorporated herein by reference.

What is claimed is:

1. A measurement device comprising:
a plurality of detection units that respectively include alight emitting unit which emits light to a measurement target site and a light receiving unit which generates detection signals corresponding to a light receiving level of the light emitted from the light emitting unit and passing through the inside of the measurement target site; and
a selection unit that selects some of the detection signals in accordance with an intensity index indicating signal intensity of the respective detection signals, from the detection signals generated by the light receiving unit in each of the plurality of detection units.

2. The measurement device according to claim 1, further comprising:
a calculation unit that calculates biological information relating to a blood flow inside the measurement target site, based on the detection signal selected by the selection unit.

3. The measurement device according to claim 1,
wherein the plurality of detection units have the same distance between the light emitting unit and the light receiving unit.

4. The measurement device according to claim 1,
wherein the plurality of detection units are installed along a first direction.

5. The measurement device according to claim 4,
wherein the light emitting unit and the light receiving unit are located along the first direction in each of the plurality of detection units.

6. The measurement device according to claim 4,
wherein the light emitting unit and the light receiving unit are located along a second direction intersecting the first direction in each of the plurality of detection units.

7. The measurement device according to claim 4,
wherein the first direction is a direction intersecting an artery inside the measurement target site.

8. The measurement device according to claim 4, further comprising:
a belt for supporting the plurality of detection units with respect to the measurement target site,
wherein the first direction is a circumferential direction of the belt.

9. The measurement device according to claim 1,
wherein the light emitted to the measurement target site from the respective light emitting units is coherent light, and
wherein a distance between the light emitting unit and the light receiving unit in each of the plurality of detection units is longer than 0.5 mm and shorter than 3 mm.

10. The measurement device according to claim 1,
wherein each of the plurality of detection units includes the plurality of light receiving units having the same distance from the light emitting unit and the light emitting unit.

11. A measurement method of measuring biological information relating to a blood flow inside a measurement target site by using a plurality of detection units respectively including a light emitting unit that emits light to the measurement target site and a light receiving unit that generates detection signals corresponding to a light receiving level of the light emitted from the light emitting unit and passing through the inside of the measurement target site, the method comprising:
causing a computer to select some of the detection signals in accordance with an intensity index indicating signal intensity of the respective detection signals, from the detection signals generated by the light receiving unit in each of the plurality of detection units; and
causing the computer to calculate the biological information, based on the selected detection signal.
